(19) United States
(12) Patent Application Publication
(10) Pub. No.: US 2023/0096850 A1
(43) Pub. Date: Mar. 30, 2023

(54) SYSTEM FOR ESTIMATING A POSE OF A SUBJECT

(71) Applicant: KONINKLIJKE PHILIPS N.V., Eindhoven (NL)

(72) Inventors: Haibo Wang, Cambridge, MA (US); Ali Shafiekhani, Missouri, MO (US); Cornelis van Zon, Everett, MA (US); Shruti Gopal Vij, Cambridge, MA (US)

(21) Appl. No.: 17/957,245

(22) Filed: Sep. 30, 2022

Related U.S. Application Data
(60) Provisional application No. 63/250,511, filed on Sep. 30, 2021.

(51) Int. Cl. G06T 7/73

(52) U.S. Cl. CPC G06T 7/74; G06T 2207/30196; G06T 2207/10016; G06T 2207/20081; G06T 2207/20084; G16H 40/20

(57) ABSTRACT

A method and a system of estimating a pose of a subject is disclosed. The method includes receiving a video stream from an imaging device in real-time; applying a trained computational model to extract feature maps from images received from the video stream; determining initial estimates of heatmaps and part affinity fields (pafs) from the extracted feature maps; refining the initial estimates of the heatmaps and pafs to output refined heatmaps and pafs; refining the heatmaps and pafs upon completing the refining of the initial estimates using a self-attention module; detecting keypoints on the heatmaps; and performing graph matching on the pafs to group the keypoints to different subjects.

This application claims the benefit of U.S. Provisional Application No. 63/250,511, filed on Sep. 30, 2021. This application is hereby incorporated by reference herein.

### BACKGROUND

The past few decades have seen substantial growth in the number of intensive care units (ICUs) and neonatal intensive care units (NICUs) worldwide. Patients who need to stay in an ICU or NICU are seriously ill and often at high risk. Therefore, ICUs and NICUs are staffed with expert teams of nurses, doctors, therapists and support staff. Family members often stay as well to provide extra support or care to the patients.

Given their condition, patients admitted to an ICU/NICU, are susceptible to adverse and catastrophic outcomes. Accordingly, it is useful to monitor not only the motions and poses of the patients, but it is also useful to monitor the activities of healthcare providers and visitors. The ICU/NICU is also an environment where invasive procedures and treatments are frequently performed, which may involve increased risk to patient safety. To reduce the risk of ICU/NICU patients, sensor-based safeguards are increasingly employed. In this context, cameras are an important sensing modality.

Certain known monitoring systems, such as camera-based systems, serve as a communication platform, and thereby remotely connect healthcare providers and patients or family members and patients. In absence of intelligent video analysis, human video monitoring and in-person “making rounds” is still needed to check on patients.

The known camera-based monitoring systems only monitor patients, but do not provide monitoring of other people in a patient's room, such as healthcare providers and visitors. Because the activities of all who enter a patient's room may be important to the care quality of patients and patient safety, the known monitoring systems are clearly deficient. Moreover, certain known systems that provide monitoring of both patients and other people in the patient's room can require significant computing resources, which increase the complexity and expense of the computer resources needed to support these known systems.

What is needed, therefore, is a method and system for monitoring the pose of a subject in that overcomes at least the drawbacks of known methods and systems described above.

### SUMMARY

According to an aspect of the present disclosure, a method of estimating a pose of a subject is disclosed. The method comprises: receiving a video stream from an imaging device in real-time; applying a trained computational model to extract feature maps from images received from the video stream; determining initial estimates of heatmaps and part affinity fields (pafs) from the extracted feature maps; refining the initial estimates of the heatmaps and pafs to output refined heatmaps and pafs; merging the heatmaps and pafs upon completing the refining of the initial estimates using a self-attention module; and performing graph matching to group keypoints to the subject.

According to another aspect of the present disclosure, a system for estimating a pose of a subject is described. The system comprises: an imaging device; a tangible, non-transitory computer readable medium adapted to stores a trained computational model comprising instructions; and a processor. The instructions, when executed by the processor, cause the processor to: apply the trained computational model to extract feature maps from images received from the video stream; determine initial estimates of heatmaps and pafs (part affinity fields) from the extracted feature maps; refine the initial estimates of the heatmaps and pafs to output refined heatmaps and pafs; further refine the heatmaps and pafs by merging them as a self-attention module; extract body keypoints on the refined heatmaps; and perform graph matching on the pafs to group the extract keypoints to different subjects.

According to another aspect of the present disclosure, a tangible, non-transitory computer readable medium that stores a computational model comprising instructions is described. When executed by a processor, the instructions cause the processor to: apply the trained computational model to extract feature maps from images received from the video stream; determine initial estimates of heatmaps and pafs (part affinity fields) from the extracted feature maps; refine the initial estimates of the heatmaps and pafs to output refined heatmaps and pafs; further refine the heatmaps and pafs by merging them as a self-attention module; extract body keypoints on the refined heatmaps; and perform graph matching on the pafs to group the extract keypoints to different subjects.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a simplified block diagram of a system for estimating a pose of a subject, according to a representative embodiment.

FIG. 2 is a simplified flow diagram showing a method for estimating a pose of a subject, according to a representative embodiment.

FIG. 3 is a flow diagram of a method of applying attention mapping to pafs image data, according to a representative embodiment.

FIG. 4 is a flow diagram of a method of applying attention mapping to heatmaps image data according to a representative embodiment.

FIG. 5 is a flow diagram showing the merging of attention mapping applied to pafs image data and heatmaps image data, according to a representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms “a,” “an” and “the” are intended to include both singular and plural forms, unless the context clearly dictates otherwise. Additionally, the terms “comprises,” “comprising,” and/or similar terms specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

By the present teachings, a imaging device (e.g., camera) based video streaming system commonly employed in clinical settings provides an intelligent monitoring platform. By contrast to known camera-based patient monitoring (e.g., vital signs camera, camera-based pressure ulcer detection), the present teachings to provide a practical application to estimate the skeleton pose of subjects in ICU/NICU to cover the monitoring of patient activity, clinical staff and family members. As alluded to above, the skeleton pose estimation of the present teachings beneficially provides the degree of resolution to ensure proper monitoring of people in a room, while requiring substantially reduced computing resources compared to known systems, resulting in a less complex and less costly system to carry out the various representative embodiments of the present teachings.

In accordance with one beneficial application, the system and methods of the present teachings are useful in identifying visitors in a patient's room, and for setting reminders for providing care to the patient. One example is that while their baby is in the Neonatal Intensive Care Unit (NICU), parents are often actively involved in the baby's care. Even the most critically ill babies will benefit from personal contact with their parents from time to time. In an effort to support this beneficial contact, the systems and methods of the various representative embodiments enable the immediate detection of the presence of a person (e.g., parent) in the room and enable monitoring of their interaction activities with the baby. The systems and methods of the present representative embodiments can provide an alert to touch or hold the newborn as needed and in a timely fashion.

In accordance with another beneficial application the system and methods of the present teachings are useful in identifying clinical staff in a patient's room, based on the monitoring of their movement, and can provide feedback of the quality and frequency of the service provided. To this end, as is known, in many clinical settings, various assessment tools are useful to account and assess performance variables that healthcare providers influence and control, while also reflecting important patient outcomes. Critical care environments typically have a multidisciplinary team approach, and the most responsible physician (MRP) usually changes on a frequent basis (with occasionally some units routinely having at least two physicians providing care daily). Thus, if a patient stays in an ICU for even just two weeks, five or six physicians and over 20 nurses could have provided care to that one patient and thus influenced their outcome. Even though ICUs practice a team-based approach to care, the opportunity for individual variations in bedside care to impact patient outcomes is very high. By the present teachings it is possible to track and examine individual clinician performance and identifying both positive and negative deviations from care protocols. As such, the systems and methods of the various representative embodiments allow the assessment of the performance of peer care groups, allowing institutions to improve care by promoting adoption of agreed-upon best practices.

To execute the desired monitoring functions, as described more fully below, the systems and methods of the present teachings track the body pose of people in a room. As described more fully below, two-dimensional (2D) pose estimation is used to localize and label a person's keypoints (i.e., joints) such as ankles, knees, hips, eyes, ears, etc. Generally, where multiple persons appear in an image, a 2D pose estimation algorithm of the representative embodiments not only localizes the keypoints, but also associates them with the individuals who appear in the image. Accordingly, extracting keypoints for each person in a single image is used as an input to other computer vision applications such as action recognition, motion capture, person identification, etc.

Beneficially, compared to known pose estimation approaches, the systems and methods of the present teachings provide multiple person 2D pose estimation while reducing the run-time complexity of inference on an edge device (CPU) and achieving the same level of accuracy compared to the known pose estimation algorithms that are either top-down or bottom-up. The former can have very high coverage rate but the latter is more efficient (i.e., runs faster). For an application like in ICU/NICU, the pose estimation approach must run with a high accuracy yet at real time. Also, for certain applications such as therapeutic care, the systems and methods of the present teachings are adapted to run on a local computer, virtual personal network, or local network, as opposed to a network cloud-based environment due to the life-critical need and concerns on network bandwidth in hospitals.

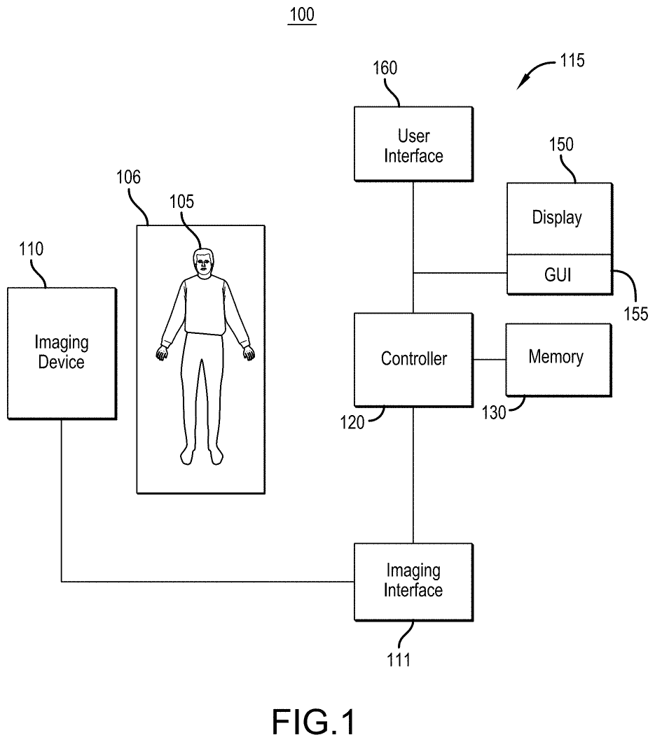

FIG. 1 is a simplified block diagram of a system 100 for estimating a pose of a subject is, according to a representative embodiment.

Referring to FIG. 1, the system 100 includes an imaging device 110 and a computer system 115 for controlling imaging of a region of interest in a patient 105 on a table 106. The imaging device 110 may be any type of medical imaging device capable of providing an image scan of the region of interest in the patient 105. In accordance with representative embodiments described below, the imaging device 110 is a camera, such as a video camera. Notably, the imaging device 110 may comprises another imaging device having an imaging modality compatible with the methods and systems of the present teachings described herein. Just by way of illustration, the imaging device 110 may comprise a sensor such as described in commonly-owned European Patent Application No. 20208465.3, filed on Nov. 18, 2020, and entitled “Device and Method for Controlling a Camera.” The disclosure of European Patent Application No. 20208465.3 is specifically incorporated herein by reference. (A copy of this incorporated document is attached.)

The computer system 115 receives image data from the imaging device 110, and stores and processes the imaging data according to the embodiments discussed herein. The computer system 115 includes a controller 120, a memory 130, a database 140 and a display 150.

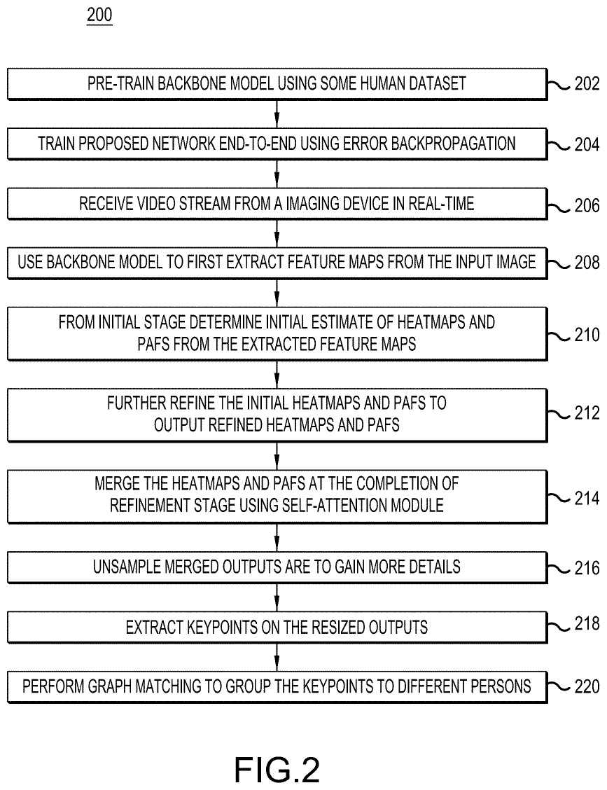

The controller 120 interfaces with the imaging device 110 through an imaging interface 111. The memory 130 stores instructions executable by the controller 120. When executed, and as described more fully below, the instructions cause the controller 120 to implement processes that include estimating a pose of a subject as described below with reference to FIGS. 2-7, for example. In addition, the controller 120 may implement additional operations based on executing instructions, such as instructing or otherwise communicating with another element of the computer system 115, including the database 140 and the display 150, to perform one or more of the above-noted processes.

The controller 120 is representative of one or more processing devices, and is configured to execute software instructions to perform functions as described in the various embodiments herein. The controller 120 may be implemented by field programmable gate arrays (FPGAs), application specific integrated circuits (ASICs), a general purpose computer, a central processing unit, a computer processor, a microprocessor, a microcontroller, a state machine, programmable logic device, or combinations thereof, using any combination of hardware, software, firmware, hard-wired logic circuits, or combinations thereof. Additionally, any processing unit or processor herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems, such as in a cloud-based or other multi-site application. The term computing device should also be interpreted to include a collection or network of computing devices each including a processor or processors. Programs have software instructions performed by one or multiple processors that may be within the same computing device or which may be distributed across multiple computing devices.

The memory 130 may include a main memory and/or a static memory, where such memories may communicate with each other and the controller 120 via one or more buses. The memory 130 stores instructions used to implement some or all aspects of methods and processes described herein. The memory 130 may be implemented by any number, type and combination of random access memory (RAM) and read-only memory (ROM), for example, and may store various types of information, such as software algorithms, which serves as instructions, which when executed by a processor cause the processor to perform various steps and methods according to the present teachings. For example, in accordance with various representative embodiments, the memory 130 that stores instructions, which when executed by the processor, cause the processor to: apply the trained computational model to extract feature maps from images received from the video stream; determine initial estimates of heatmaps and pafs (part affinity fields) from the extracted feature maps; refine the initial estimates of the heatmaps and pafs to output refined heatmaps and pafs; further refine the heatmaps and pafs by merging them as a self-attention module; extract body keypoints on the refined heatmaps; and perform graph matching on the pafs to group the extract keypoints to different subjects. Notably, as is known, in pose estimation, heatmaps are used to locate anatomical features such as joints, and pafs are used to estimate connections between the anatomical features.

More generally, after being trained, the computational model may be stored as executable instructions in memory 130, for example, to be executed by a processor of the controller 120. Furthermore, updates to the computational model may also be provided to the computer system 115 and stored in memory 130. Finally, and as will be apparent to one of ordinary skill in the art having the benefit of the present disclosure, according to a representative embodiment, the computational model may be stored in a memory and executed by a processor that are not part of the computer system 115, but rather is connected to the imaging device 110 through an external link (e.g., a known type of internet connection). Just by way of illustration, the computational model may be stored as executable instructions in a memory, and executed by a server that is remote from the imaging device 110. When executed by the processor in the remote server, the instructions cause the processor apply the trained computational model to extract feature maps from images received from the video stream; determine an initial estimate of heatmaps and pafs from the extracted feature maps; refine the initial estimate of the heatmaps and pafs to output refined heatmaps and pafs; merge the heatmaps and pafs upon completing the refinement of the initial estimates using a self-attention module; and perform graph matching to group keypoints to the subject.

The various types of ROM and RAM may include any number, type and combination of computer readable storage media, such as a disk drive, flash memory, an electrically programmable read-only memory (EPROM), an electrically erasable and programmable read only memory (EEPROM), registers, a hard disk, a removable disk, tape, compact disk read only memory (CD-ROM), digital versatile disk (DVD), floppy disk, Blu-ray disk, a universal serial bus (USB) drive, or any other form of storage medium known in the art. The memory 130 is a tangible storage medium for storing data and executable software instructions, and is non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The memory 130 may store software instructions and/or computer readable code that enable performance of various functions. The memory 130 may be secure and/or encrypted, or unsecure and/or unencrypted.

Similarly, the database 140 stores data and executable instructions used to implement some or all aspects of methods and processes described herein. Notably, the database 140 can be foregone, and all data and executable instructions can be stored in memory 130.

The database 140 may be implemented by any number, type and combination of RAM and ROM, for example, and may store various types of information, such as software algorithms, AI models including RNN and other neural network based models, and computer programs, all of which are executable by the controller 120. The various types of ROM and RAM may include any number, type and combination of computer readable storage media, such as a disk drive, flash memory, EPROM, EEPROM, registers, a hard disk, a removable disk, tape, CD-ROM, DVD, floppy disk, Blu-ray disk, USB drive, or any other form of storage medium known in the art. The database 140 is a tangible storage medium for storing data and executable software instructions that are non-transitory during the time software instructions are stored therein. The database 140 may be secure and/or encrypted, or unsecure and/or unencrypted.

“Memory” and “database” are examples of computer-readable storage media, and should be interpreted as possibly being multiple memories or databases. The memory 130 or database 140 may, for instance, be multiple memories or databases local to the computer, and/or distributed amongst multiple computer systems or computing devices. Furthermore, the memory 130 and the database 140 comprise a computer readable storage medium that is defined to be any medium that constitutes patentable subject matter under 35 U.S.C. § 101 and excludes any medium that does not constitute patentable subject matter under 35 U.S.C. § 101.

The controller 120 illustratively includes or has access to an AI engine, which may be implemented as software that provides artificial intelligence (e.g., a bottom-up human pose estimation) and applies machine-learning. The AI engine, which provides a computational model described below, may reside in any of various components in addition to or other than the controller 120, such as the memory 130, the database 140, an external server, and/or a cloud, for example. When the AI engine is implemented in a cloud, such as at a data center, for example, the AI engine may be connected to the controller 120 via the internet using one or more wired and/or wireless connection(s). The AI engine may be connected to multiple different computers including the controller 120, so that the artificial intelligence and machine-learning described below in connection with various representative embodiments are performed centrally based on and for a relatively large set of medical facilities and corresponding subjects at different locations. Alternatively, the AI engine may implement the artificial intelligence and the machine-learning locally to the controller 120, such as at a single medical facility or in conjunction with the imaging device 110, which may be a single imaging device.

The interface 160 may include a user and/or network interface for providing information and data output by the controller 120 and/or the memory 130 to the user and/or for receiving information and data input by the user. That is, the interface 160 enables the user to enter data and to control or manipulate aspects of the processes described herein, and also enables the controller 120 to indicate the effects of the user's control or manipulation. The interface 160 may include one or more of ports, disk drives, wireless antennas, or other types of receiver circuitry. The interface 160 may further connect one or more user interfaces, such as a mouse, a keyboard, a mouse, a trackball, a joystick, a microphone, a video camera, a touchpad, a touchscreen, voice or gesture recognition captured by a microphone or video camera, for example.

The display 150 may be a monitor such as a computer monitor, a television, a liquid crystal display (LCD), a light emitting diode (LED) display, a flat panel display, a solid-state display, or a cathode ray tube (CRT) display, or an electronic whiteboard, for example. The display 150 may also provide a graphical user interface (GUI) 155 for displaying and receiving information to and from the user.

FIG. 2 is a simplified flow diagram showing a method 200 for imaging blood flow, according to a representative embodiment. Various aspects and details of the method 200 are implemented using the system 100 in accordance with representative embodiments described below. Certain details of the system 100 and may not be repeated in order to avoid obscuring the discussion of the present representative embodiment.

At 202, the method begins with pre-training a basic feature extraction computational model. Generally, this pre-training is carried out using a feature extraction model as a Backbone model known to those of ordinary skill in the art. In accordance with a representative embodiment, the pre-training algorithm is a modified version of a “lightweight” convolutional neural network known as ShuffleNet V2 to provide the basic feature extraction module. Further details of the ShuffleNet V2 algorithm that is modified in accordance with various representative embodiments may be found in “ShuffleNet V2: Practical Guidelines for Efficient CNN Architecture Design” (Ningning Ma, Xiangyu Zhang, Hai-Tao Zheng, Jian Sun, CVPR 2018), the disclosure of which is specifically incorporated herein by reference (a copy of this document is attached). It is emphasized that the use of Shufflenet V2 is merely illustrative, and other similar (or even more lightweight) convolutional neural network models could be used in the embodiment.

Compared to its counterparts like VGG net, ResNet and MobileNet, ShuffleNet V2 is particularly useful by providing a low footprint usage on system 100, and in particular, the controller 120. As such, the trained feature extraction computational model of 202 beneficially provides a comparatively high inference speed yet requires fewer computing resources to known feature extraction methods. In accordance with a representative embodiment, the ShuffleNet V2 algorithm is modified by adding a dilation operation in the inverted residual module to accommodate non-rigid body appearance variations. Dilated convolutions inflate the convolution kernel by inserting holes between the kernel elements. The insertion is controlled by a dilation factor such as 2, 4, or 8. Since it involves pixel skipping, it covers a larger area of input feature map, which in turn potentially could handle various appearance variations. Further details of the noted dilation operation may be found, for example, in “Dilated Residual Networks” Fisher Yu, Vladlen Koltun, Thomas Funkhouser; (Proceedings of the IEEE Conference on Computer Vision and Pattern Recognition (CVPR), 2017, pp. 472-480.) The entire disclosure of “Dilated Residual Networks” is specifically incorporated by reference herein, and a copy of this document is attached to the present filing.

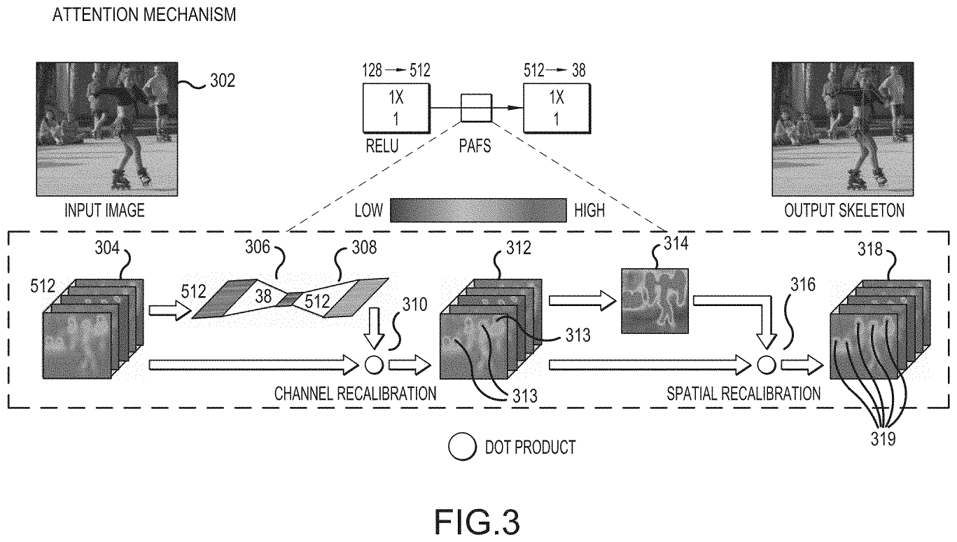

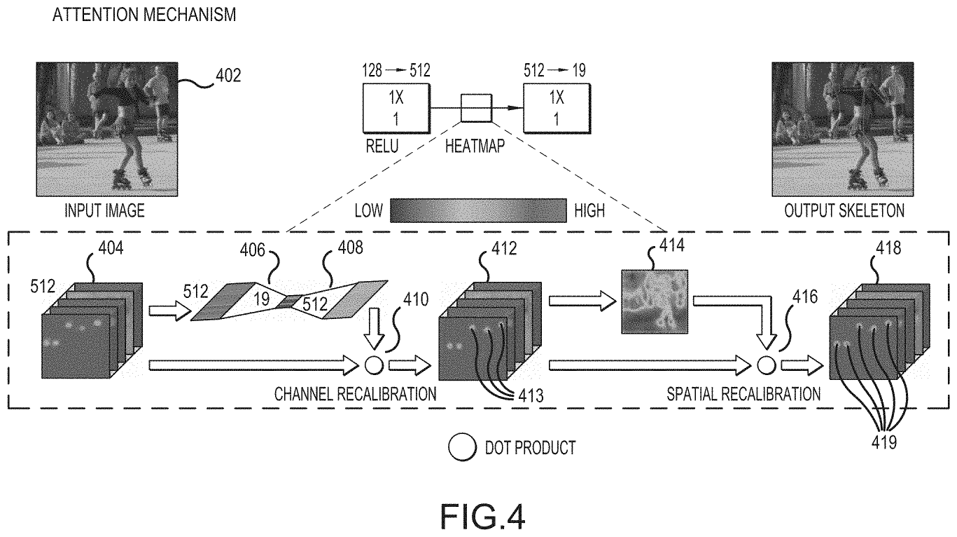

The trained computational model of the representative embodiments provides further modification to the modified ShuffleNet backbone by adding refinement stages. As described more fully below in connection with FIGS. 3 and 4, these refinement stages comprise regular feature convolutions and pooling, but enhanced by first, adding spatial and channel wise attention, and second changing the structure of network from a two-branch to a single branch network. As a result, and just by way of illustration, the inference time required may decrease by a factor of 2.5 compared to a known multiple person 2D pose estimation algorithm for edge devices. This known multiple person 2D pose estimation algorithm may be described, for example in “Monocular human pose estimation: A survey of deep learning-based methods, Computer Vision and Image Understanding, Volume 192, March 2020” (Yucheng Chen, Yingli Tian and Mingyi He), the disclosure of which is specifically incorporated herein by reference. (A copy of this document is attached). At 204, the entire system 100, which may be part of a network, is trained end-to-end using error backpropagation. The training sequence of the computational model is carried out using one of a number of known machine-learning techniques known to those of ordinary skill in the art of AI and mathematical models. Just by way of illustration, the backbone model (e.g., the above-referenced modified ShuffleNet) could be first pretrained and then fine-tuned together with the rest of the system end-to-end.

In machine-learning, an algorithmic model “learns” how to transform its input data into meaningful output. During the learning sequence, the computational model adjusts its inner parameters given the input parameters of the examples, and produces the corresponding meaningful output or so-called target. The adjustment process is guided by instructions on how to measure the distance between the currently produced output and the desired output. These instructions are called the objective function.

In deep learning, which is a subfield of machine-learning, the inner parameters inside the computational model are organized into successively connected layers, where each layer produces increasingly meaningful output as input to the next layer, until the last layer which produces the final output.

Deep learning layers are typically implemented as so-called neural networks, that is, layers are comprised of a set of nodes, each representing an output value and a prescription on how to compute this output value from the set of output values of the previous layer's nodes. The prescription being a weighted sum of transformed output values of the previous layer's nodes, each node only needs to store the weights. The transformation function is the same for all nodes in a layer and is also called activation function. There are a limited number of activation functions that are used today. A particular way to set which previous layer's nodes provide input to a next layer's node is convolution. Networks based on this way are called convolutional neural networks.

Thus, in the learning phase or so-called training, for each example, the output of the final layer is computed. Outputs for all examples are compared with the desired outputs by way of the objective function. The output of the objective function, the so-called loss, is used as a feedback signal to adjust the weights of all the previous layers one-by-one. The adjustment, i.e. which weights to change and by how much, is computed by the central algorithm of deep learning, so-called backpropagation, which is based on the fact that the weighted sums that connect the layer nodes are functions that have simple derivatives. The adjustment is iterated until the loss reaches a prescribed threshold or no longer changes significantly.

A deep-learning network thus can be stored (e.g., in database 140 or memory 130) as a topology that describes the layers and activation functions and a (large) set of weights (simply values). A trained network is the same, only the weights are now fixed to particular values. Once the network is trained, it is ready to use, that is, to predict output for new input for which the desired output is unknown.

In the present teaching, the computational model is stored as instructions to provide the machine-learning algorithm, such as a convolutional neural-network algorithm. When executed by a processor, the machine-learning algorithm (the computational model) of the representative embodiments is used to provide two-dimensional (2D) pose estimation that localizes and labels a person's keypoints (i.e., joints) such as ankles, knees, hips, eyes, ears, etc and their connections on each input image.

Once the computational model is trained, at 206 a video stream from the imaging device 110 is received by the controller 120. Generally, this video streaming is continuously run.

At 208, the feature extraction model, which is illustratively a Backbone model, extracts feature maps from the input image. As described more fully below, the feature maps are used to map the regions of interest/importance that are useful in the ultimate pose estimation. Beneficially, compared to the original input image, the feature maps extracted at 208 provide more distinctive pose related information while less important pixels are suppressed.

At 210 the initial heatmaps and pafs provide an initial estimate of the locations of the desired anatomical elements. The initial stage is to provide a first-round of heatmaps and pafs. Notably, the first-round of heatmaps and pafs may be obtained through multiple rounds of convolutions and pooling operations similar to the Openpose process described, for example in 1 “OpenPose: Realtime Multi-Person 2D Pose Estimation using Part Affinity Fields” (Zhe Cao, et al. IEEE Transactions on Pattern Analysis and Machine Intelligence (Volume: 43, Issue: 1, Jan. 1, 2021) The entire disclosure of “OpenPose: Realtime Multi-Person 2D Pose Estimation using Part Affinity Fields is specifically incorporated by reference, and a copy of this document is attached.

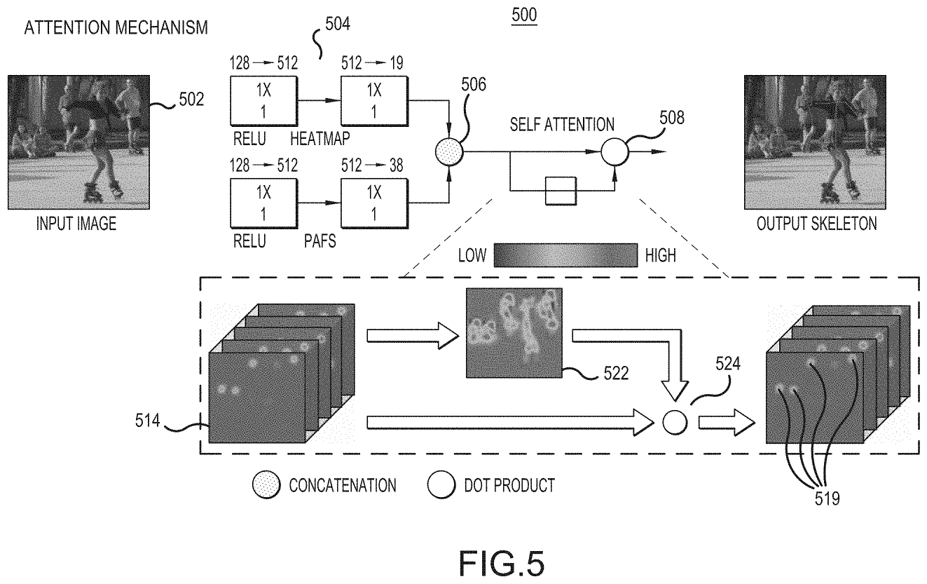

At 212 the initial heatmaps and pafs are refined. As described more fully below, these refinements may be done simultaneously, with the heatmaps and pafs merged into a final refined image with emphasis on the desired features of the body. Notably, and as described more fully below, the refinement of the heatmaps and pafs provides enhanced images at a pixel level to provide the desired features, while requiring less computing power by the controller 120, and less data storage in the memory. Ultimately, the desired degree of resolution of the estimated poses is achieved while requiring less computing power. As will be appreciated, this provides estimated poses accurately, but more quickly and at a lower cost. At 214, the heatmaps and pafs are merged using a self-attention map sequence as described more fully below in connection with FIG. 5. This allows the heatmaps and pafs to iteratively refine each other. The self-attention map boosts the refinement as heatmaps and pafs are found is mutually beneficial. Notably, the merged image provides the desired resolution of the important features (e.g., anatomical joints and the connections therebetween) using less computing power and memory by not mapping unimportant features to the refined image.

216-220 provide post-processing of the refined images and provide the desired skeleton poses of the subjects. At 216, the merged heatmaps from 214 are unsampled to provide greater details of the desired important features from the refined image. Specifically at 216, the output heatmaps are unsampled to the same size of the original input image so as to provide more accurate keypoint locations at the original image.

At 218, keypoints are extracted on the resized heatmaps. By way of illustration, keypoint detection approaches implemented at 218 may be as described in the above-incorporated reference to Cao, et al. At a testing phase, body parts are first predicted on the resized heatmaps. Keypoints are then defined at the maximum feature response by performing non-maximum suppression on neighboring areas.

Finally, at 220, the keypoints of the individual subject(s) are grouped via graph matching. For a single subject, keypoint association is directly yield on the output pafs. For the general case of multiple subjects, a K-partite graph of the keypoint associations of all subjects is first built based on the geometrical distances of joints. It is then relaxed into a set of bipartite graphs, the optimal matching of which could be solved together via the Hungarian algorithm. More details and how to handle the case of multiple subjects could be found in the above-incorporated reference to Cao, et al.

FIG. 3 is a simplified flow diagram of a method 300 of applying attention mapping to pafs image data, according to a representative embodiment. Notably, various aspects of the method 300 are implemented using the system 100 and the method 200 described above. Common details and features of the system 100 and the method 200 may not be repeated to avoid obscuring the description of the presently described representative embodiments. An input image 302 is provided and pafs maps 304 are extracted and provided as a plurality of channels (e.g., 512). At 306, an initial feature extraction model (e.g., a backbone model) is applied the pafs map comprising the greater number (e.g., 512) channels to map the features to a smaller number (e.g., 38) channels thereby reducing data requirements and processing by the controller 120. The channel attention map exploits the inter-channel relation of features to focus on what is meaningful given an input image. Channel attention is computed by squeezing the spatial dimension of the input feature map by using both average-pooling and maxing-pooling. More details about how the channel attention is mapped to input channels may be found in “Squeeze-and-Excitation Networks” (2018 IEEE/CVF Conference on Computer Vision and Pattern Recognition, to Jie Hu, et al.). This mapping step, which is referred to as channel attention mapping, may be carried out using the modified Shufflenet feature extraction algorithm described above. Notably, as designated by the dot product shown in FIG. 3, a channel recalibration step 310 is provided to each pixel of each of the of the channels by a application of a weighting function based on the relative importance of the desired anatomical feature. This results in an initially refined pafs map multi-channel having pixels deemed “important” (i.e., pixels of a desired anatomical feature) enhanced, and those deemed less important in the current mapping having reduced intensity. Notably, in the representative embodiment currently described, the “important” pixels are of the face and head 313. As will be appreciated, while the images are presented over a larger number of channels (e.g., 512), because only facial features are emphasized, less data is required and lower processing requirements result if the less important pixels remained at the same intensity as at the input.

A merged spatial attention map on pafs 314 is provided as shown, and includes emphasized pixels from the multi-channel initially refined pafs maps 312.

At 316 a spatial recalibration step is applied to the pafs 314. As designated by the dot product shown in FIG. 3, the channel recalibration step 316 is provided to each pixel of each of the of the channels from the multi-channel initially refined pafs map 312 by mapping the emphasized pixels to from the pafs 314 by an application of a weighting function based on the relative importance of the desired anatomical feature. This results in a multi-channel initially refined pafs map 312 further refined pafs to provide an output 318 having the desired “important” pixels 319 (e.g., connections between joints) having an increased intensity. Again, while the images are presented over a larger number of channels (e.g., 512), because only facial features (in this example) are emphasized, less data is required and lower processing requirements result if the less important pixels remained at the same intensity as at the input.

FIG. 4 is a simplified flow diagram of a method 400 of applying attention mapping to heatmaps image data according to a representative embodiment. Notably, various aspects of the method 400 are implemented using the system 100 and the methods 200, 300 described above. Common details and features of the system 100 and the methods 200, 300 may not be repeated to avoid obscuring the description of the presently described representative embodiments.

An input image 402 is provided and heatmap 404 are extracted and provided as a plurality of channels (e.g., 512). At 406, an initial feature extraction model (e.g., a backbone model) is applied the heatmap comprising the greater number (e.g., 512) of channels to map the features to a smaller number (e.g., 38) of channels thereby reducing data requirements and processing by the controller 120. The channel attention map exploits the inter-channel relation of features to focus on what is meaningful given an input image. Channel attention is computed by squeezing the spatial dimension of the input feature map by using both average-pooling and maxing-pooling. More details about how the channel attention is mapped to input channels may be found in the above-incorporated document to Jie Hu, et al. This mapping step, which is referred to as channel attention mapping, may be carried out using the modified Shufflenet feature extraction algorithm described above. Notably, as designated by the dot product shown in FIG. 4, a channel recalibration step 410 is provided to each pixel of each of the channels by an application of a weighting function based on the relative importance of the desired anatomical feature. This results in a multi-channel initially refined heatmap 412 having pixels deemed “important” (i.e., pixels of a desired anatomical feature) enhanced, and those deemed less important in the current mapping having reduced intensity. Notably, in the representative embodiment currently described, the “important” pixels are of the face and head 413. As will be appreciated, while the images are presented over a larger number of channels (e.g., 512), because only facial features are emphasized, less data is required and lower processing requirements result if the less important pixels remained at the same intensity as at the input.

A spatial attention map 414 is provided as shown, and includes emphasized pixels 415 squeezed from the multi-channel initially refined heatmaps 412.

At 416 a spatial recalibration step is applied to the spatial attention map 414. As designated by the dot product shown in FIG. 4, the spatial recalibration step is provided to each pixel of each of the channels from the multi-channel initially refined heatmap 412 by mapping the emphasized pixels to from the spatial attention map 414 by an application of a weighting function based on the relative importance of the desired anatomical feature. This results in a multi-channel initially refined heatmap 412 further refined to provide an output 418 having the desired “important” pixels (e.g., the face and head 419) having an increased intensity. Again, while the images are presented over a larger number of channels (e.g., 512), because only facial features (in this example) are emphasized, less data is required and lower processing requirements result if the less important pixels remained at the same intensity as at the input.

FIG. 5 is a simplified flow diagram of a method 500 of refining pafs and heatmaps by iteratively merging them to perform self-attention mapping, according to a representative embodiment. Notably, various aspects of the method 500 are implemented using the system 100 and the methods 200, 300, 400 described above. Common details and features of the system 100 and the methods 200, 300, 400 may not be repeated to avoid obscuring the description of the presently described representative embodiments.

FIG. 5 illustrates how pafs and heatmaps are refined each other via the self-attention mechanism. As in 506, all the paf and heatmap channels (e.g., 19+38->57 channels) are concatanated. Next, at 508 a spatial self-attention map is constructed by applying spatial max-pooling on all the pixels of all the concatenated feature channels. Finally, at 510 the spatial self-attention map is applied back to each of the concatenated channels via dot product. This results in the transformation of the pafs and heatmap 514 to the channel attention map 522. As such, a channel recalibration step is applied to the pafs and heatmap 514. As designated by the dot product shown in FIG. 5, a spatial recalibration step is provided to each pixel of each of the channels from the multi-channel initially refined pafs and heatmap 514 by mapping the emphasized pixels to from the pafs and heatmap 514 by an application of a weighting function based on the relative importance of the desired anatomical feature. This self-attention mapping sequence is effective in producing a pose estimations because pafs and heatmaps mutually benefit each other, such as, for example, enabling part association to aid in emphasizing body joint locations and vice versa. This results in a multi-channel initially refined heatmap further refined to provide an output 518 having the desired “important” pixels (e.g., the face and head 519) having an increased intensity. Again, while the images are presented over a larger number of channels (e.g., 512), because only facial features (in this example) are emphasized, less data is required and lower processing requirements result if the less important pixels remained at the same intensity as at the input.

As will be appreciated by one of ordinary skill in the art having the benefit of the present disclosure, the systems and methods of the present teachings provide improvement in the function of an imaging system used to improve monitoring of people in a patient's room or other similar area, and an improvement to the technical field of monitor imaging. For example, compared to known methods and systems, accurate pose estimation and movement of people is realized by the present teachings, at a comparatively high inference speed yet requiring fewer computing resources to known feature extraction methods. Notably, the benefits are illustrative, and other advancements in the field of pose estimation and monitoring of people will become apparent to one of ordinary skill in the art having the benefit of the present disclosure.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Although methods, systems and components for estimating a pose of a subject have been described with reference to several exemplary embodiments, it is understood that the words that have been used are words of description and illustration, rather than words of limitation. Changes may be made within the purview of the appended claims, as presently stated and as amended, without departing from the scope and spirit of pose estimation of a subject in its aspects. Although developing adaptable predictive analytics has been described with reference to particular means, materials and embodiments, developing adaptable predictive analytics is not intended to be limited to the particulars disclosed; rather developing adaptable predictive analytics extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A method of estimating a pose of a subject, the method comprising:
receiving a video stream from an imaging device in real-time;
applying a trained computational model to extract feature maps from images received from the video stream;
determining initial estimates of heatmaps and part affinity fields (pafs) from the extracted feature maps;
refining the initial estimates of the heatmaps and pafs to output refined heatmaps and pafs;
merging the heatmaps and pafs upon completing refining of the initial estimates using a self-attention module; and
performing graph matching to group keypoints to the subject.

2. The method of claim 1, further comprising, after the merging, unsampling the merged heatmaps and pafs.

3. The method of claim 1, wherein the trained computational model comprises a bottom-up trained computational model.

4. The method of claim 3, wherein the bottom-up trained computational model comprises a human pose estimation computational model.

5. The method of claim 1, further comprising applying a feature extracting computational model.

6. The method of claim 5, wherein the feature extracting computational model comprises a Backbone computational model.

7. The method of claim 1, wherein the imaging device comprises a camera.

8. The method of claim 1, wherein the imaging device comprises a sensor device.

9. A system for estimating a pose of a subject, the system comprising:
an imaging device;
a tangible, non-transitory computer readable medium adapted to stores a trained computational model comprising instructions; and
a processor, wherein the instructions, when executed by the processor, cause the processor to:
apply the trained computational model to extract feature maps from images received from a video stream;
determine an initial estimate of heatmaps and part affinity fields (pafs) from the extracted feature maps;
refine the initial estimate of the heatmaps and pafs to output refined heatmaps and pafs;
merge the heatmaps and pafs upon completing the refinement of the initial estimates using a self-attention module; and
perform graph matching to group keypoints to the subject.

10. The system of claim 9, wherein the instructions further cause the processor to unsample the merged heatmaps and pafs.

11. The system of claim 9, wherein the trained computational model comprises a bottom-up trained computational model.

12. The system of claim 11, wherein the bottom-up trained computational model comprises a human pose estimation computational model.

13. The system of claim 9, wherein the instructions, when executed by the processor, cause the processor to apply a feature extracting computational model to extract the heatmaps and pafs.

14. The system of claim 9, wherein the imaging device comprises a camera, or a sensor, or both.

15. A tangible, non-transitory computer readable medium that stores instructions for a trained computational model, wherein the instructions, when executed by a processor, cause the processor to:
apply the trained computational model to extract feature maps from images received from a video stream;
determine an initial estimate of heatmaps and part affinity fields (pafs) from the extracted feature maps;
refine the initial estimate of the heatmaps and pafs to output refined heatmaps and pafs;
further refine the heatmaps and pafs upon completing the refinement of the initial estimates using a self-attention module;
extract body keypoints on the refined heatmaps; and
perform graph matching on the refined pafs to group keypoints to a subject.

16. The tangible, non-transitory computer readable medium of claim 15, wherein the instructions further cause the processor to unsample merged heatmaps and pafs.

17. The tangible, non-transitory computer readable medium system of claim 15, wherein wherein the trained computational model comprises a bottom-up trained computational model.

18. The tangible, non-transitory computer readable medium system of claim 17, wherein the bottom-up computational model comprises a human pose estimation computational model.

19. The tangible, non-transitory computer readable medium of claim 15, wherein the instructions further cause the processor to apply a feature extracting computational model to extract the heatmaps and pafs.

20. The tangible, non-transitory computer readable medium of claim 19, wherein the feature extracting computational model comprises a Backbone computational model.
